(19) United States
(12) Patent Application Publication
(10) Pub. No.: US 2022/0144670 A1
(43) Pub. Date: May 12, 2022

(54) HANDHELD SANITIZING DEVICE

(71) Applicant: Carson Young, West Bountiful, UT (US)

(72) Inventor: Carson Young, West Bountiful, UT (US)

(21) Appl. No.: 17/094,797

(22) Filed: Nov. 10, 2020

(51) Int. Cl. C02F 1/32; A61L 2/10

(52) U.S. Cl. CPC C02F 1/325; A61L 2/10; A61L 2202/11; A61L 2202/16; C02F 2201/008; C02F 2301/024; C02F 2307/02; C02F 2201/009; A61L 2202/23

(57) ABSTRACT

A handheld sanitizing device may include a housing with a first end and a second end. The housing may enclose a first cavity and a second cavity. A sanitizing light may be disposed at the first end of the housing. The sanitizing light may be configured to kill biological contaminants. A removable cap may attach to the first end and may cover the sanitizing light. An extendable agitator may extend from the first end of the housing and retract into the first cavity. A motor disposed in the first cavity may actuate the agitator. The agitator may be configured to stir a fluid. The housing may be configured to sit in a mouth of a container, extend into a reservoir of the container, and rest on the mouth of the container. A charging port may be disposed on the housing and electronically coupled to the motor and/or the sanitizing light.

### BACKGROUND

Various substances and/or surfaces may become contaminated by biological contaminants such as viruses, bacteria, and/or parasites. Such biological contaminants may cause a human or other animal, such as a pet or domesticated animal, to become ill. In some cases, the malady resulting from a biological contaminant may cause death. For example, some water sources such as streams, rivers, ponds, and/or lakes may contain bacteria such as E. Coli and/or parasites such as Giardia. As another example, surfaces may transfer, by contact, various viral contaminants, such as the flu virus. Transfer of biological contaminants to humans and/or other animals may present not only a personal health risk but also a public health risk. For example, various regions may experience shortages of clean drinking water and/or contamination of previously potable water. Access to clean drinking water may also be limited in regions remote from populated areas, such as in wilderness areas. People may be discouraged from exploring or traveling to such areas when potable water is not accessible.

### BRIEF DESCRIPTION OF THE DRAWINGS

The present description will be understood more fully when viewed in conjunction with the accompanying drawings of various examples of handheld sanitizing devices. The description is not meant to limit the handheld sanitizing devices to the specific examples. Rather, the specific examples depicted and described are provided for explanation and understanding of handheld sanitizing devices. Throughout the description the drawings may be referred to as drawings, figures, and/or FIGs.

FIG. 1A illustrates a perspective view of a handheld sanitizing device, according to an embodiment.

FIG. 1B illustrates a front view of the handheld sanitizing device with a removable cap removed, exposing a sanitizing light and an extendable agitator, according to an embodiment.

FIG. 1C illustrates the extendable agitator extended from a housing of the handheld sanitizing device, according to an embodiment.

FIG. 2A illustrates the handheld sanitizing device inserted into a container, according to an embodiment.

FIG. 2B illustrates the handheld sanitizing device with a container adapter, according to an embodiment.

FIG. 3 illustrates a cross-section of the handheld sanitizing device, according to an embodiment.

FIG. 4A illustrates the handheld sanitizing device with separate switches for the sanitizing light and a motor, according to an embodiment.

FIG. 4B illustrates the handheld sanitizing device with an automatic switch for the motor, according to an embodiment.

FIG. 5A illustrates the handheld sanitizing device with the extendable agitator off-center from a center of a first end of the handheld sanitizing device, according to an embodiment.

FIG. 5B illustrates an extendable arm of the extendable agitator coupled off-center to an agitator end of the extendable agitator, according to an embodiment.

FIG. 5C illustrates a head-on view of the first end of the handheld sanitizing device where the agitator end is nested in the sanitizing light, according to an embodiment.

FIG. 6A illustrates the extender arm extending directly from the motor, according to an embodiment.

FIG. 6B illustrates the extender arm coupled to a rotor of the motor by a linear translation mechanism, according to an embodiment.

### DETAILED DESCRIPTION

Handheld sanitizing devices as disclosed herein will become better understood through a review of the following detailed description in conjunction with the figures. The detailed description and figures provide merely examples of the various embodiments of handheld sanitizing devices. Many variations are contemplated for different applications and design considerations; however, for the sake of brevity and clarity, all the contemplated variations may not be individually described in the following detailed description. Those skilled in the art will understand how the disclosed examples may be varied, modified, and altered and not depart in substance from the scope of the examples described herein.

A conventional water purification device may include a water container, a pump, and a filter. The pump may draw unpurified water through the filter and into the water container. However, water filters require significant physical exertion and only neutralize some contaminants, leaving many contaminants in the water. In some cases, the water is pumped through the filter several times to make it drinkable, requiring significant time and physical exertion to purify the water.

Another conventional water purification system may include a water container and a dissolvable tablet. The dissolvable tablet may include a compound such as dichloroisocyanurate which, when dissolved in water, may neutralize micro-organisms in water. However, a minimum amount of time is necessary for the compound to be effective, sometimes up to half an hour. Additionally, if the correct amount of compound is not used, the water may not be sufficiently purified or may be rendered undrinkable because of too much compound.

Another water purification device may include an ultraviolet light. The ultraviolet light may include various wavelengths of light at intensities sufficient to kill micro-organisms in the water. However, the ultraviolet light may not be exposed to all parts of the liquid, leaving some contaminants in the liquid. The ultraviolet light may require batteries, and if no batteries are available, or if the batteries that power the ultraviolet light are dead, the light cannot be used to purify the water. Additionally, current implementations of the ultraviolet light are inconvenient and clunky, often being dropped into water containers or otherwise not well-fit to a water container. This leads to incomplete purification, leaving the water unsafe to drink.

Implementations of handheld sanitizing devices described below may address some or all of the problems described above. A handheld sanitizing device may include an elongated housing with a first end and a second end opposite the first end. A first cavity in the elongated housing may be adjacent to the first end and a second cavity in the elongated housing may be adjacent to the second end. A sanitizing light may be disposed at the first end facing away from the elongated housing. The sanitizing light may be configured to kill biological contaminants. A removable cap may attach to the first end and cover the sanitizing light. An extendable agitator may extend from the first end of the elongated housing and retract into the first cavity. A motor may be disposed in the first cavity and operably connected to the agitator. The agitator may be configured to stir a liquid substance as the motor actuates the agitator.

The elongated housing may have a first diameter around the first cavity that is configured to fit within a mouth of a container. The elongated housing may have a second diameter around the second cavity that is greater than a diameter of the mouth of the container. The elongated housing may be configured to sit in the mouth of the container, extend into a reservoir of the container, and rest on the mouth of the container to prevent the sanitizing device from falling into the reservoir. A charging port may be disposed on the second end of the elongated housing and electronically coupled to the motor and/or the sanitizing light. The charging port may directly provide power to the electronic components of the sanitizing device and/or may charge a battery that powers the electronic components.

Handheld sanitizing devices as described herein may address at least some of the problems described above regarding previous water purification devices and systems. For example, the handheld sanitizing device may be configured to fit conveniently in water bottles and rest on the mouth of a water bottle to prevent the sanitizing device from falling in the water bottle. The agitator may stir water in the water bottle as the sanitizing light shines into the water, which may increase exposure of the sanitizing light to contaminants in the water. This arrangement is more likely to achieve sufficient water purification to render the water drinkable. In at least some cases, this may enable the sanitizing device to neutralize all or substantially all biological contaminants in the water. This may also substantially decrease the amount of time to purify the water.

The charging port may enable the sanitizing device to be powered by a ubiquitous device that people already carry with them everywhere: a cell phone or other mobile electronic device. The handheld sanitizing devices described herein may, when used, increase accessibility to clean water. For example, individuals hiking into remote areas may use the handheld sanitizing device to purify water from lakes, rivers, streams, ponds, pools, and so forth.

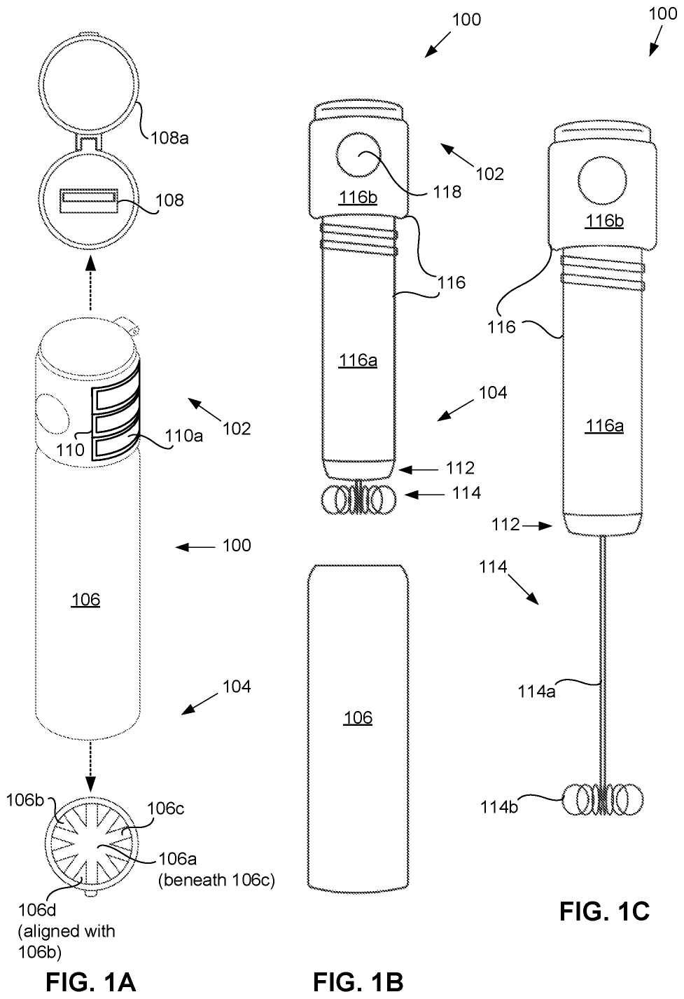

FIG. 1A illustrates a perspective view of a handheld sanitizing device 100, according to an embodiment. The handheld sanitizing device 100 may have a compact profile that allows the handheld sanitizing device 100 to be conveniently carried. Additionally, the handheld sanitizing device 100 may be powered by an external device, such as a mobile phone or other mobile electronic device. The handheld sanitizing device 100 may be used to sanitize substances for consumption, such as water. The handheld sanitizing device 100 may be used to sanitize surfaces, such as surfaces in public areas where many people touch the surfaces. Thus, the handheld sanitizing device 100 may help protect against biological contaminants and/or micro-organisms that may cause a person to become ill.

The handheld sanitizing device 100 may include a first end 102 and a second end 104. The first end 102 may be opposite the second end 104. The handheld sanitizing device 100 may have a greater length than width, where the first end 102 and the second end 104 are disposed at opposite ends of the handheld sanitizing device 100 along the length of the handheld sanitizing device 100 (i.e. the first end 102 may be positioned opposite the second end 104 lengthwise on the handheld sanitizing device 100). The handheld sanitizing device 100 may be approximately cylindrical. The handheld sanitizing device 100 may be approximately a rectangular cuboid. The handheld sanitizing device 100 may be approximately prismatic (i.e. may have a triangular width-wise cross-section). The handheld sanitizing device 100 may have an approximately uniform width along the length of the handheld sanitizing device 100. The handheld sanitizing device 100 may have different widths along the length of the handheld sanitizing device 100. The handheld sanitizing device may have a width that tapers outwards and/or inwards from the first end 102 to the second end 104. The handheld sanitizing device 100 may have different widths at the opposing ends of the handheld sanitizing device 100.

A removable cap 106 may cover the first end 102 of the handheld sanitizing device 100. The removable cap 106 may extend at least a portion of the length of the handheld sanitizing device 100 from the first end 102 to the second end 104. The removable cap 106 may attach to the first end 102 of the handheld sanitizing device 100. The removable cap 106 may attach to a body of the handheld sanitizing device 100. The removable cap 106 may extend over the body of the handheld sanitizing device 100 along the length of the handheld sanitizing device 100. The removable cap 106 may extend approximately one-tenth of the length of the handheld sanitizing device 100, approximately one-fifth the length of the handheld sanitizing device 100, approximately one-quarter the length of the handheld sanitizing device 100, approximately one-third the length of the handheld sanitizing device 100, approximately one-half the length of the handheld sanitizing device 100, approximately two-thirds the length of the handheld sanitizing device 100, approximately three-quarters the length of the handheld sanitizing device 100, approximately four-fifths the length of the handheld sanitizing device 100, approximately nine-tenths the length of the handheld sanitizing device 100, approximately an amount between one-tenth and nine-tenths of the length of the handheld sanitizing device 100, and so forth.

The removable cap 106 may include a portion that actuates to reveal and re-cover a sanitizing light on the body of the handheld sanitizing device 100. The portion may be twistable. The portion may be hinged with a flap. The portion may be slidable. The portion may enable the sanitizing light to be revealed without removing the removable cap 106. For example, the sanitizing light may be used to sanitize a surface. The actuatable portion of the removable cap 106 may enable the sanitizing light to be exposed without exposing other portions of the handheld sanitizing device 100 to the environment. This may ensure that portions of the handheld sanitizing device 100 that will be inserted into a drinking bottle remain relatively sanitary while still enabling the handheld sanitizing device 100 to be used for sanitizing surfaces.

As an example of the actuatable portion of the removable cap 106, the removable cap 106 may include, approximately parallel to the width dimension of the handheld sanitizing device 100, a first layer 106a. The first layer 106a may include a first opening or set of openings 106b that aligns with the sanitizing light as the removable cap 106 is attached to the body of the handheld sanitizing device 100. The removable cap 106 may include, approximately parallel to the first layer 106a, a second layer 106c. The second layer 106c may be inside the removable cap 106 or outside the removable cap 106. The second layer 106c may include a second opening or set of openings 106d that corresponds to the first opening or set of openings 106b. The first layer 106a and the second layer 106c may be rotatably coupled to each other. In a first position of the second layer 106c relative to the first layer 106a, the second opening 106d may be aligned with the first opening 106b so that the sanitizing light is exposed. In a second position of the second layer 106c relative to the first layer 106a, the second layer 106c may cover the first opening 106b so that the sanitizing light is covered.

The handheld sanitizing device 100 may include a power port 108. The power port 108 may be disposed at the second end 104 of the handheld sanitizing device 100 opposite the sanitizing light. The power port 108 may be configured to connect with an end of a power cable. For example, the power port 108 may be a universal serial bus type A (USB-A) port, a USB-B port, a USB-C port, a mini USB port, a micro USB port, a Lightning® port, and so forth. The power port 108 may be a male end or a female end. The power port 108 may deliver power to various electronic components of the handheld sanitizing device 100, such as the sanitizing light, an internal controller, a motor, a rechargeable battery, and so forth. The power port 108 may deliver power to the electronic component(s) from an external power source. For example, the power port 108 may be connected to a power cable that is connected to a smartphone. The power port 108 may deliver power from the smartphone via the power cable to the electronic component(s). The power port 108 may be configured to transmit data to the controller. For example, the power port 108 may transmit configuration instructions from the smartphone to the controller.

The handheld sanitizing device 100 may include a lid 108a that covers the power port 108. The lid 108a may be hinged or may be removable from the handheld sanitizing device 100. The lid 108a may be an insert that inserts into the power port 108. The lid 108a may be a screw-on lid.

The handheld sanitizing device 100 may include a solar panel 110. The solar panel 110 may be physically coupled to the body of the handheld sanitizing device 100. The solar panel 110 may be physically coupled to the removable cap 106. The solar panel 110 may include a photosensitive surface 110a. The photosensitive surface 110a may face outwards from the handheld sanitizing device 100. The solar panel 110 may be electronically coupled to one or more electronic components of the handheld sanitizing device 100, such as the sanitizing light, the motor, or the controller, the rechargeable battery, and so forth. The solar panel 110 may be configured to directly power the electronic component(s) of the handheld sanitizing device 100.

FIG. 1B illustrates a front view of the handheld sanitizing device 100 with the removable cap 106 removed, exposing a sanitizing light 112 and an extendable agitator 114, according to an embodiment. The sanitizing light 112 and the extendable agitator 114 may be used to purify (i.e. sanitize) a liquid, such as water. The water may have been obtained from a source that is known to be contaminated with biological contaminants such as viruses, bacteria, and/or parasites. The water may have been obtained from a source where the purity of the water is unknown.

The sanitizing light 112 may kill such biological contaminants. The extendable agitator 114 may stir the water to ensure that all or a substantial portion of the water is exposed to the sanitizing light 112. This may speed up the purification process and instill confidence that the water is purified. This may also reduce user error which might otherwise result in incomplete purification. For example, the extendable agitator 114 may cycle the water through the path of the sanitizing light 112 at a sufficiently high rate that outpaces a user's impatience to complete the purification process. In some cases, a 16-ounce bottle of water may be purified in under 30 seconds. Larger water containers suitable for individual use may be purified in only a few minutes, as opposed to a half-hour or more for previous purification systems.

The handheld sanitizing device 100 may include a housing 116. The housing 116 may be elongated such that a length of the housing 116 is greater than a width of the housing 116. The housing 116 may define a shape of the handheld sanitizing device 100. The housing 116 may define at least a portion of the first end 102 and/or at least a portion of the second end 104. The sanitizing light 112 may be disposed in and/or on the housing 116. For example, the sanitizing light 112 may be mounted to the first end 102 of the handheld sanitizing device 100 defined by the housing 116. The sanitizing light 112 may be mounted in or on the housing 116 such that the sanitizing light 112 emits light rays away from the housing 116. The extendable agitator 114 may similarly be mounted in or on the housing 116. For example, at least a portion of the extendable agitator 114 may be positioned in the housing 116 and at least another portion of the extendable agitator 114 may be positioned outside the housing 116. The extendable agitator may be positioned adjacent to, and/or extend through, the first end 102.

The housing may be formed of one or more materials, such as plastic, rubber, metal, and so forth. For example, at least a portion of the housing 116 may be made of a thermoplastic formed in a mold or extruded. The housing 116 may be monolithic (i.e. a single piece) or may include two or more pieces connected together. The pieces may be bonded together or joined together so that the pieces can be separated without damaging the pieces (e.g. the separate pieces are joined by a catch-and-hook mechanism, and so forth). A first segment 116a of the housing 116 may be separable from a second segment 116b of the housing 116. The first segment 116a may be screwed to the second segment 116b. The first segment may include the first end 102, and the second segment 116b may include the second end 104.

The sanitizing light 112 may be configured to at least partially sanitize a substance or a surface. The sanitizing light 112 may be incandescent, fluorescent, luminescent, and so forth. For example, the sanitizing light 112 may be a light-emitting diode (LED). The LED may emit one or more wavelengths of light. The sanitizing light 112 may emit wavelengths from the short-UV to the near-infrared. Such wavelengths may kill and/or inactivate gram-positive bacteria, gram-negative bacteria, fungi, endospores, parasites, viruses, protein toxins, and so forth. For example, the sanitizing light 112 may emit light having one or more wavelengths in a range from 200 nanometers (nm) to 400 nm, from 300 nm to 550 nm, from 500 nm to 700 nm, from 650 nm to 800 nm, and so forth. The range of wavelengths may be selectable. This may be useful to target specific types of micro-organisms. For example, a water source may be known to be contaminated with a specific parasite. The range of wavelengths emitted by the sanitizing light 112 may be selected for the specific parasite. The sanitizing light 112 may include a single light source or two or more light sources (e.g. two or more LEDs). The sanitizing light 112 may include different light sources tuned to different wavelengths. The sanitizing light 112 may be configured to output the light at a sufficiently high power rating to kill and/or inactivate biological contaminants. For example, the sanitizing light 112 may emit light with a power in a range from 1 micro-Watt to 1 milli-Watt, from 1 milli-Watt to 1 Watt, and so forth.

The removable cap 106 may attach to the first end 102 of the handheld sanitizing device 100, e.g. the first segment 116a of the housing 116. The removable cap 106 may cover the sanitizing light 112 and/or the extendable agitator 114. The removable cap 106 may include a ridge/wall 106e that attaches the removable cap 106 to the housing 116. For example, the removable cap 106 may include a relatively short ridge that extends a relatively short distance along the length of the housing 116, or a longer wall that extends a relatively long distance along the length of the housing 116. The wall may extend approximately the entire length of the first segment 116a of the housing. The removable cap 106 may attach to the housing 116 by snap-fit. The removable cap 106 and the housing 116 may include corresponding threading, and the removable cap 106 may screw onto the housing 116. The removable cap 106 may be attached to the housing 116 by a hinge and may swing away from the housing 116 when removed to expose the sanitizing light 112 and/or the extendable agitator 114.

The handheld sanitizing device 100 may include a switch 118. The switch 118 may switch power to the electronic component(s) of the handheld sanitizing device 100 on and/or off. The switch may be a push-button. The push-button may be concave or convex. The push-button may extend out and away from an outside surface of the housing 116. The push-button may be approximately flush with the outside surface of the housing 116. The push-button may be recessed into the housing 116. The switch 118 may be a toggle switch. The switch 118 may be a slider. A position of the push-button, the toggle switch, and/or the slider may indicate an operative status of an electronic component of the handheld sanitizing device (i.e. on, off, and so forth). The switch 118 may enable switching between wavelengths of light for the sanitizing light 112 and/or various speeds for the extendable agitator 114. For example, a first press of the push-button may turn the sanitizing light 112 on to a first wavelength, a second press of the push-button may switch the sanitizing light 112 to a different wavelength, and so forth. A first press of the push-button may turn the extendable agitator 114 on to a first speed, a second press of the push-button may switch the extendable agitator 114 to a different speed, and so forth. A first position of the toggle switch and/or slider may turn the sanitizing light 112 on to a first wavelength, a second position of the toggle switch and/or slider may switch the sanitizing light 112 to a different wavelength, and so forth. A first position of the toggle switch and/or slider may turn the extendable agitator 114 on to a first speed, a second position of the toggle switch and/or slider may switch the extendable agitator 114 to a different speed, and so forth.

FIG. 1C illustrates the extendable agitator 114 extended from the housing 116 of the handheld sanitizing device 100, according to an embodiment. The extendable agitator 114 may extend from the housing 116 to improve mixing of fluid in a container and/or to reach into containers with deep reservoirs. For example, for a 16-ounce water bottle, the housing 116 may have a length that reaches to approximately ⅓ of the depth of the reservoir. The extendable agitator 114, when extended from the housing 116, may further reach to half the depth of the reservoir, ⅔ of the depth of the reservoir, and so forth. By reaching further into the reservoir, the extendable agitator 114 may enable stirring of water at the bottom of the reservoir. The handheld sanitizing device 100 may be pulled up and down in the reservoir to achieve stirring of all the water in the reservoir. The extendable agitator 114 may reach to half the depth of the reservoir and may, without pulling up and down in the reservoir, cause enough mixing to stir a substantial portion and/or all of the water in the reservoir. This may enable a substantial portion and/or all of the water in the reservoir to be purified by the sanitizing light 112.

The extendable agitator 114 may be configured to extend from and retract towards the housing 116. For example, the extendable agitator 114 may be configured to extend from the first end 102 and/or the first segment 116a of the housing 116. The extendable agitator 114 may extend automatically, such as by a motor that extends and retracts the extendable agitator 114. The extendable agitator 114 may extend manually, such as by being pulled out and pushed back in by hand or some other external mechanism external to the handheld sanitizing device 100.

The extendable agitator 114 may include an extender arm 114a and an agitator end 114b. The extender arm 114a may be extendable from and retractable into the housing 116. The agitator end 114b may be coupled to the extender arm 114a. The agitator end may include one or more surfaces, shapes, and/or designs that, when moved in a fluid, cause mixing of and/or turbulence in the fluid.

For example, the agitator end may include one or more rings. The rings may be attached to the extender arm 114a such that a plane formed by a single ring is approximately coplanar with, and/or parallel to, a length dimension of the extender arm 114a. When the agitator end 114b spins, fluid may pass through an open portion of the ring and the edges of the ring may create turbulence in the fluid. The turbulence may lead to mixing of the fluid. As another example, the agitator end may include blades that are tilted, similar to an arrangement of fan blades. As another example, the agitator end may include blades positioned so that the surfaces of the blades with the greatest surface area is approximately perpendicular to the direction of rotation of the agitator end 114b. The surfaces of the blades may push the fluid such that mixing is induced when the agitator end 114b spins in the fluid.

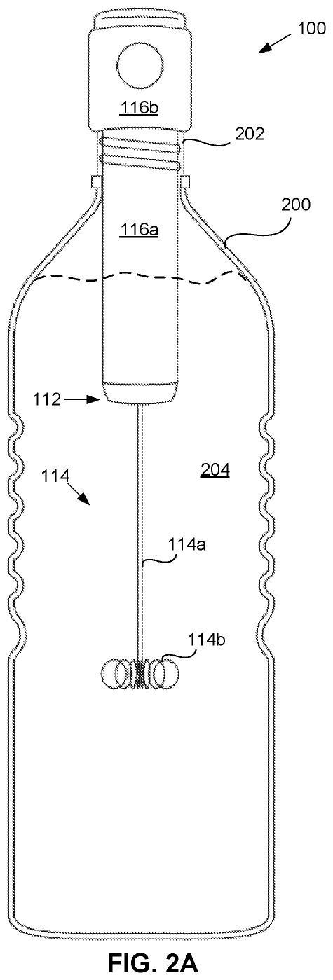

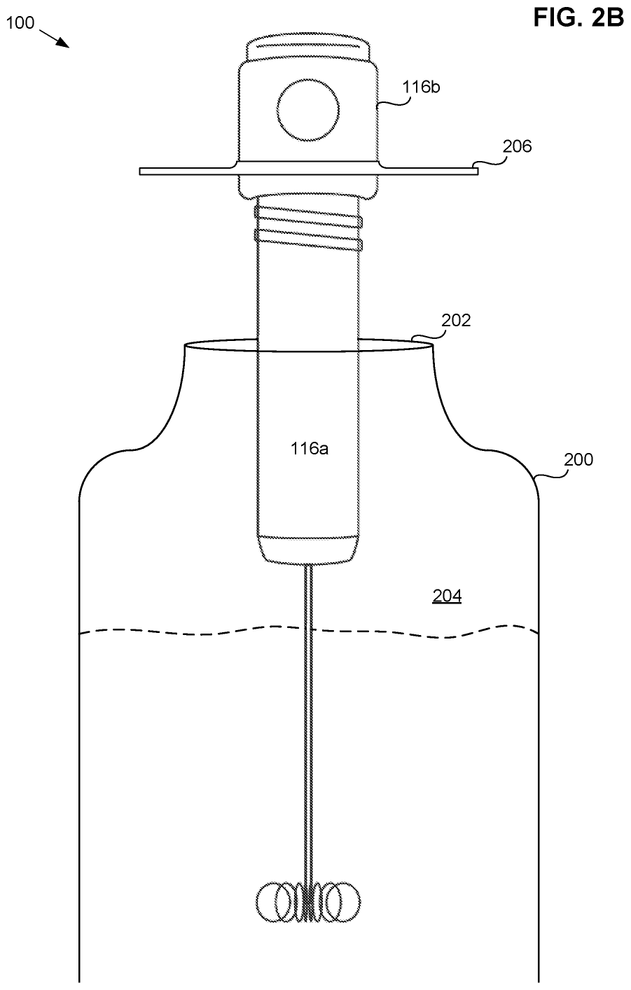

FIG. 2A illustrates the handheld sanitizing device 100 inserted into a container 200, according to an embodiment. The handheld sanitizing device 100 may be sized such that the first segment 116a of the housing 116 fits through a mouth 202 of the container 200 and the second segment 116b of the housing 116 rests against the mouth 202 of the container. This may help prevent a user from dropping the handheld sanitizing device 100 into the container 200. This may also make the handheld sanitizing device 100 easier to use. For example, a user may be able to hold the container 200 and the handheld sanitizing device 100 together with one hand, leaving the user's other hand free. This may further allow the container 200 to be shaken while the handheld sanitizing device 100 is in the container 200 without spilling water from the container 200. Shaking the container 200 may improve mixing of the water within the container and help achieve more complete purification of the water.

The container 200 may include the mouth 202 and a reservoir 204. The reservoir may hold a fluid, such as water. The housing 116 may be configured to pass at least partially through the mouth 202 of the container 200 and into the reservoir 204 of the container 200. For example, the diameter of the first segment 116a may be less than the diameter of the mouth 202, and the diameter of the second segment 116b may be greater than the diameter of the mouth 202. The extendable agitator 114 may extend into the fluid and stir the fluid as the agitator end rotates (e.g. as a motor in the handheld sanitizing device 100 rotates the extendable agitator 114. The sanitizing light 112 may kill, inactivate, and/or otherwise neutralize biological contaminants in the fluid as the extendable agitator stirs the fluid.

The diameter of the first segment 116a of the housing 116 may be in a range from half an inch to one inch, from five-eighths of an inch to seven-eighths of an inch, or approximately three-quarters of an inch, and so forth. The diameter of the second segment 116b of the housing 116 may be in a range from half an inch to one-and-a-half inches, from five-eighths of an inch to one-and-three eighths of an inch, from three-quarters of an inch to one-and-a-quarter of an inch, from seven-eighths of an inch to one-and-one-eighth of an inch, or approximately one inch, and so forth. The diameter dimensions are not intended to be strictly limiting, but, rather, merely examples for illustrative purposes.

FIG. 2B illustrates the handheld sanitizing device 100 with a container adapter 206, according to an embodiment. In some cases, the handheld sanitizing device 100 may be used with a container 200 that has a mouth 202 with a greater diameter than the diameter of the first segment 116a and the second segment 116b. The container adapter 206 may enable the handheld sanitizing device 100 to be inserted into the container 200, rest against the mouth 202 of the container 200, and/or seal the reservoir 204 of the container 200 so that the container 200 may be shaken. The container adapter 206 may also render the handheld sanitizing device 100 usable with containers having various mouth widths.

The container adapter 206 may be configured to attach to the housing 116. The container adapter may include an approximately planar surface that forms a ring around an opening. The opening may have a diameter that is approximately the same as a diameter of the housing 116. The housing 116 may fit in the opening and form a frictional engagement with inner edges of the ring that define the opening of the container adapter 206. The frictional engagement may hold the container adapter 206 to the housing 116 and/or may prevent the handheld sanitizing device 100 from slipping out of the opening of the container adapter 206. The container adapter 206 may snap-fit onto the housing 116. The container adapter 206 may be otherwise attached to the housing 116. The container adapter 206 may have a diameter that is greater than the diameter of the housing 116 and/or the mouth 202 of the container 200. The container adapter 206 may rest against the mouth 202 of the container 200 and prevent the handheld sanitizing device 100 from falling into the container 200.

In some examples, the handheld sanitizing device 100 may be used in conjunction with another purifying method. For example, a purifying tablet may be placed into the container 200. The handheld sanitizing device 100 may be inserted into the container 200. The extendable agitator 114 may stir the liquid (e.g. water) in the container 200 as the tablet dissolves and the sanitizing light 112 shines UV light into the container 200. The extendable agitator 114 may speed-up the dissolving process of the tablet while simultaneously stirring the liquid to draw liquid into the path of the UV light.

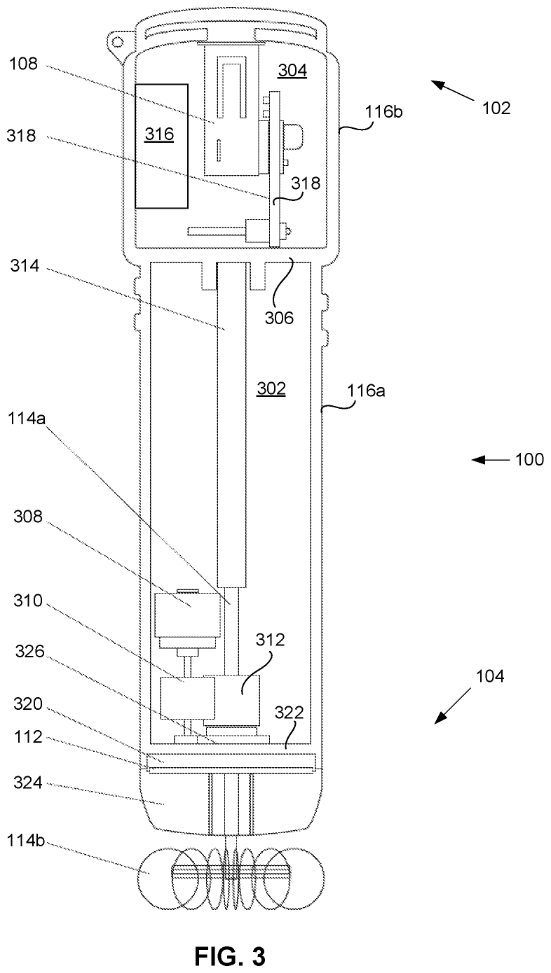

FIG. 3 illustrates a cross-section of the handheld sanitizing device 100, according to an embodiment. The housing 116 main include internal cavities that may contain electronic components of the handheld sanitizing device 100. The electronic components may enable the handheld sanitizing device 100 to stir and/or sanitize a fluid. The housing 116 may be water-tight and may isolate the internal electronic components of the handheld sanitizing device 100 from fluids as portions of the handheld sanitizing device are inserted into the fluids.

The housing 116 may define a first cavity 302. The housing 116 may define a second cavity 304. The first cavity 302 may be adjacent to the first end 102 of the handheld sanitizing device 100. The second cavity 304 may be adjacent to the second end 104 of the handheld sanitizing device 100. The first cavity 302 may be defined by the first segment 116a of the housing 116. The second cavity 304 may be defined by the second segment 116b of the housing 116. The first cavity 302 and the second cavity may be separated from each other by an internal wall 306 of the housing 116.

The sanitizing light 112 may be disposed on the housing 116 adjacent to the first cavity 302. The sanitizing light 112 may be disposed adjacent to the first cavity 302 at the first end 102 of the handheld sanitizing device 100. The power port 108 may be disposed on the housing 116 adjacent to and/or within the second cavity 304. The sanitizing light 112 may face away from the housing 116 such that the sanitizing light 112 shines light away from the housing 116. The power port 108 may be disposed adjacent to and/or within the second cavity 304 adjacent to the second end 104 of the handheld sanitizing device 100. The extendable agitator 114 may be disposed within and/or adjacent to the first cavity 302. For example, the extender arm 114a may be disposed within the first cavity 302 as the extender arm 114a is retracted into the first cavity 302. The agitator end may be disposed approximate to the first cavity 302 at the first end 102 of the handheld sanitizing device 100. The extender arm 114a may be disposed at least partially within the first cavity 302 and adjacent to the first end 102 as the extender arm 114a is extended from the housing 116.

A motor 308 may be disposed within the first cavity 302. The motor 308 may be mounted to (e.g. coupled to) an interior surface of the housing 116 that defines the first cavity 302. The motor 308 may be operatively coupled to the extender arm 114a to rotate the extendable agitator 114. For example, the motor 308 may include a rotor that directly spins a gear 310. The gear 310 may engage with the extender arm 114a and/or a cuff 312 that directly engages with the extender arm 114a. The cuff 312 may, for example, be frictionally engaged with the extender arm 114a. The cuff 312 and the extender arm 114a may have corresponding catches that engage when the cuff 312 is rotated by the gear 310, transferring the rotation to the extender arm 114a. The extender arm 114a may be engaged with the cuff 312 such that the extender arm 114a may linearly translate (i.e. slide through) the cuff 312 while still being sufficiently engaged that rotation of the cuff 312 translates into rotation of the extender arm 114a. The extender arm may include gear teeth that directly engage with the gear 310. A support shaft 314 may be disposed in the first cavity 302. The support shaft may be coupled to the internal wall 306 between the first cavity 302 and the second cavity 304. The support shaft 314 may be at least partially hollow. The support shaft 314 may support the extender arm 114a to ensure the extender arm 114a remains straight and/or does not disengage from the motor 308. The extender arm 114a may telescope into and from the support shaft 314. In another example, the extender arm 114a may be at least partially hollow and the support shaft 314 may be positioned at least partially within the extender arm 114a.

A battery 316 may be disposed within the first cavity 302 and/or the second cavity 304. The battery 316 may be mounted to the interior surface of the housing 116 and/or to the internal wall 306. The battery 316 may be rechargeable. The battery 316 may be rechargeable via the power port 108. The battery 316 may power one or more electronic components of the handheld sanitizing device 100, such as the sanitizing light 112, the motor 308, and so forth. In various examples of the handheld sanitizing device 100, the battery 316 may be excluded. The electronic components of the handheld sanitizing device 100 may be powered directly via the power port 108 without the battery 316 being present in and/or on the handheld sanitizing device 100. For example, the power port 108 may be electronically coupled to the sanitizing light 112, the motor 308, and/or the controller 318. The power port 108 may directly power the sanitizing light 112, the motor 308, and/or the controller 318.

A controller 318 may be disposed within the first cavity 302 and/or the second cavity 304. The controller 318 may be electronically coupled to the power port 108, the sanitizing light 112, the motor 308, the switch 118, the battery 316, and so forth. For example, the controller 318 may be electronically coupled to the power port 108, the sanitizing light 112, the motor 308, the switch 118, and/or the battery 316 by wiring and/or electrical tracing. The controller 318 may include firmware and/or software for controlling the power port 108, the sanitizing light 112, the motor 308, the switch 118, and/or the battery 316. For example, the controller 318 may include firmware and/or software that determines which wavelength of light is emitted by the sanitizing light 112 based on, for example, a position and/or number of presses of the switch 118. The controller 318 may include firmware and/or software that regulates the speed of the motor based on, for example, a position and/or number of presses of the switch 118.

The controller 318 may be programmable. For example, the controller 318 may reset one or more settings of the controller 318 based on instructions received from a device separate from the handheld sanitizing device 100. The instructions may be received via the power port 108. The controller 318 may include a wireless communication device that enables the controller 318 to receive instructions and/or settings from the device that is separate from the handheld sanitizing device 100. The wireless communication device may include a Bluetooth® chip, a WiFi chip, a near-field communications (NFC) chip, a long-range (LoRa) communication chip, and so forth. The wireless communication device may enable various of the electronic components of the handheld sanitizing device 100 to be controlled by the device that is separate from the handheld sanitizing device 100. For example, a smartphone may control the wavelength of light and/or the intensity of light emitted by the sanitizing light 112 via the controller 318 and the wireless communication device. The smartphone may control the speed of the motor 308 via the controller 318 and the wireless communication device. The smartphone may control the sanitizing light 112 and/or the motor 308 via the power port 108.

The sanitizing light 112 may be coupled to a printed circuit board (PCB) 320. The PCB 320 may include control electronics (i.e. electronic hardware) for the sanitizing light 112. The PCB 320 may be mounted to an external wall 322 that defines at least a portion of the first cavity 302 at the first end 102 of the handheld sanitizing device 100. The handheld sanitizing device 100 may include a lens 324 over the sanitizing light 112. The lens 324 may be attached to the housing 116 at the first end 102 of the handheld sanitizing device 100. The lens 324 may focus and/or diffuse light emitted by the sanitizing light 112.

The external wall 322, the PCB 320, and/or the lens 324 may include openings through which the extender arm 114a of the extendable agitator 114 may pass. The openings may be concentric with a centerline axis of the handheld sanitizing device 100. The openings may be off-center from (i.e. not aligned with) the centerline axis. A seal 326 may be disposed in one or more of the openings. The seal 326 may form a liquid-tight (e.g. water-tight) seal between the extender arm 114a and the housing 116, the lens 324, and/or the PCB 320. The extender arm 114a may be rotatable as the seal 326 form the liquid-tight seal with the extender arm 114a.

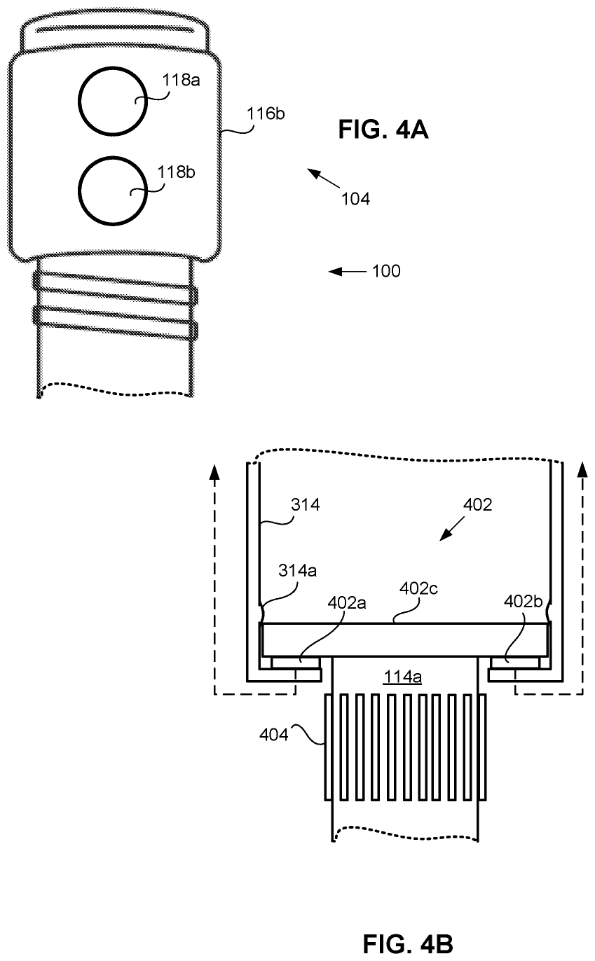

FIG. 4A illustrates the handheld sanitizing device 100 with separate switches for the sanitizing light 112 and the motor 308, according to an embodiment. In some cases, the sanitizing light 112 may be utilized without the extendable agitator 114, such as for sanitizing surfaces. The extendable agitator 114 may remain retracted into and/or adjacent to the housing 116 as the sanitizing light 112 is used to sanitize the surface. Separate switches for the sanitizing light 112 and the motor 308 may enable the sanitizing light 112 to be used without the extendable agitator 114 being rotated. This may prevent damage by the agitator end 114b to components of the handheld sanitizing device 100 such as the housing 116, the lens 324, the sanitizing light 112, and so forth. This may also allow the sanitizing light 112 to be brought close to the surface without damaging the surface.

The handheld sanitizing device 100 may include a first switch 118a and a second switch 118b. The first switch 118a may control power to the sanitizing light 112. The second switch 118b may control power to the motor 308. The first switch 118a and the second switch 118b may be pressed simultaneously to turn on the sanitizing light 112 and the motor 308 at the same time. The first switch 118a and the second switch 118b may be pressed separately to power the sanitizing light 112 and the motor 308, respectively, separately.

In some examples of the handheld sanitizing device 100, the switch 118 may include different positions that control power to the sanitizing light 112 and the motor 308. In a first position of the switch 118, the sanitizing light 112 may be on and the motor 308 may be off. In a second position of the switch 118, the motor 308 may be on and the sanitizing light 112 may be off. In a third position of the switch 118, the sanitizing light 112 and the motor 308 may both be on. In a fourth position of the switch 118, the sanitizing light 112 and the motor 308 may both be off.

FIG. 4B illustrates the handheld sanitizing device 100 with an automatic switch for the motor 308, according to an embodiment. In at least some examples, the switch 118 may power the sanitizing light 112 and the motor 308. The extendable agitator 114 may be configured to rotate when extended and not rotate when retracted. This may simplify operation of the handheld sanitizing device 100, having a single switch for both the sanitizing light 112 and the motor 308, and ensuring the extendable agitator 114 does not rotate when retracted towards the housing 116.

The handheld sanitizing device 100 may be configured such that the motor 308 receives power when the extendable agitator 114 is extended. The handheld sanitizing device 100 may be configured such that the motor 308 does not receive power when the extendable agitator is not extended. The motor 308 may receive power as the extendable agitator 114 is extended a threshold length from the housing 116. The motor 308 may not receive power as the extendable agitator 114 is retracted within the threshold length towards the housing 116. The extendable agitator 114 may be configured to engage with the motor 308 as the extendable agitator 114 is extended from the housing 116. The extendable agitator 114 may be configured to be disengaged from the motor 308 as the extendable agitator 114 is retracted into the housing 116. In at least some examples, the support shaft 314 may include catches 314a that retain the extendable agitator 114 in the extended position at and/or beyond the threshold length from the housing 116.

The handheld sanitizing device 100 may include an automatic switch 402 that engages when the extendable agitator 114 is extended the threshold length. For example, the automatic switch 402 may include a first contact 402a, a second contact 402b, and a bridge contact 402c. The first contact 402a and the second contact 402b may, for example, be mounted to an interior of the support shaft 314. The first contact 402a and the second contact 402b may further be electronically coupled to the controller 318 and/or the motor 308. The bridge contact 402c may be mounted to an end of the extender arm 114a. When the extendable agitator 114 is extended the threshold distance, the bridge contact 402c may form electrical contact with the first contact 402a and the second contact 402b to close the automatic switch 402. When the automatic switch 402 is closed, the motor 308 may receive power to rotate the extendable agitator 114.

The extender arm 114a of the extendable agitator 114 may include a gear surface 404. The gear surface 404 may, for example, engage with the cuff 312 and/or the gear 310. When the extendable agitator 114 is extended the threshold distance from the housing 116, the gear surface 404 may engage with the cuff 312 and/or the gear 310. Rotation of the rotor of the motor 308 may be translated to rotation of the extendable agitator via the gear 310, the cuff 312, and/or the gear surface 404. In some examples of the handheld sanitizing device 100, the support shaft 314 may include windings and the extender arm 114a may include one or more magnets. The windings may be electronically coupled to the controller 318, the power port 108, the battery 316, and so forth. When the magnets in the extender arm 114a align within the windings, such as when the extendable agitator 114 is extended the threshold length from the housing 116, the extendable agitator 114 may rotate.

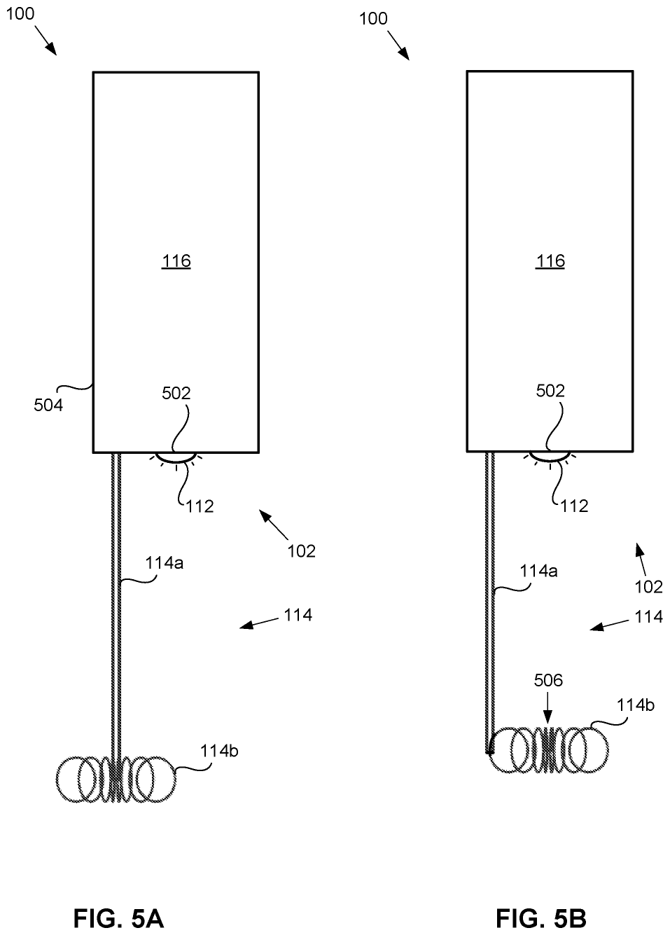

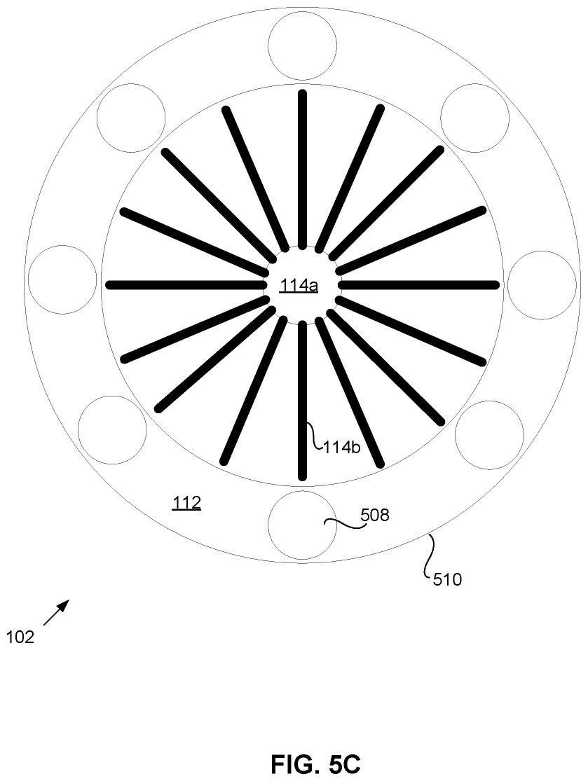

FIG. 5A illustrates the handheld sanitizing device 100 with the extendable agitator 114 off-center from a center 502 of the first end 102, according to an embodiment. Having the extendable agitator 114 off-center may put the agitator end 114b close to a sidewall of the container 200 when the handheld sanitizing device 100 is inserted into the container 200. This may increase an amount of mixing and/or turbulence created by the agitator end 114b as the extendable agitator 114 is rotated. Increased mixing and/or turbulence may lead to faster and/or more complete purification of the fluid being stirred by the extendable agitator 114.

The sanitizing light 112 may be disposed in approximately the center 502 of the first end 102 of the handheld sanitizing device 100. The center 502 may refer to, for example, a center of a surface of the housing 116 at the first end 102. The extender arm 114a may be positioned adjacent to the sanitizing light 112 and off-center from the center 502 of the first end 102. As the extendable agitator 114 is retracted into the housing 116, the extender arm may be positioned along a side of the housing 116 such as adjacent to a side wall 504 of the housing.

FIG. 5B illustrates the extendable arm 114a coupled off-center to the agitator end 114b, according to an embodiment. Attaching the extendable arm 114a off-center to the agitator end 114b may increase an effective radius of the agitator end 114b as the extendable agitator 114 is rotated by the motor 308. Having an increased radius may further increase mixing and/or turbulence in the fluid being stirred by the extendable agitator 114. Increased mixing and/or turbulence may lead to faster and/or more complete purification of the fluid being stirred by the extendable agitator 114.

The agitator end 114b may include a ring. The agitator end 114b may, as the extendable agitator 114 is retracted towards the housing 116 and/or as the agitator end is adjacent to the first end 102 of the housing, encircle the sanitizing light 112. The extender arm 114a may be coupled to the agitator end 114b off-center from a center 506 of the agitator end 114b.

In various examples, the sanitizing light 112 may be positioned on the agitator end 114b. For example, individual LEDs may be positioned on individual blades of the agitator end 114b. Power may be delivered to the sanitizing light by an electrical conduit (e.g. an insulated wire) in and/or on the extender arm 114a.

FIG. 5C illustrates a head-on view of the first end 102 of the handheld sanitizing device 100 where the agitator end 114b is nested in the sanitizing light 112, according to an embodiment. The sanitizing light 112 may be used to sanitize surfaces in addition to purifying fluids such as water. The sanitizing light 112 may be exposed such that the agitator end 114b does not block light emitted from the sanitizing light 112 from reaching the surface to be sanitized. Nesting the agitator end 114b in the sanitizing light 112 may allow for a direct path for the light from the sanitizing light 112 to the surface.

The sanitizing light 112 may be formed in a ring configuration (e.g. a ring light). Emitters 508 of the sanitizing light 112 may be positioned adjacent to an edge 510 of the housing 116 at the first end 102 of the handheld sanitizing device 100. The agitator end 114b of the extendable agitator 114 may nest within the sanitizing light 112 as the agitator end 114b is adjacent to the first end 102 of the handheld sanitizing device 100.

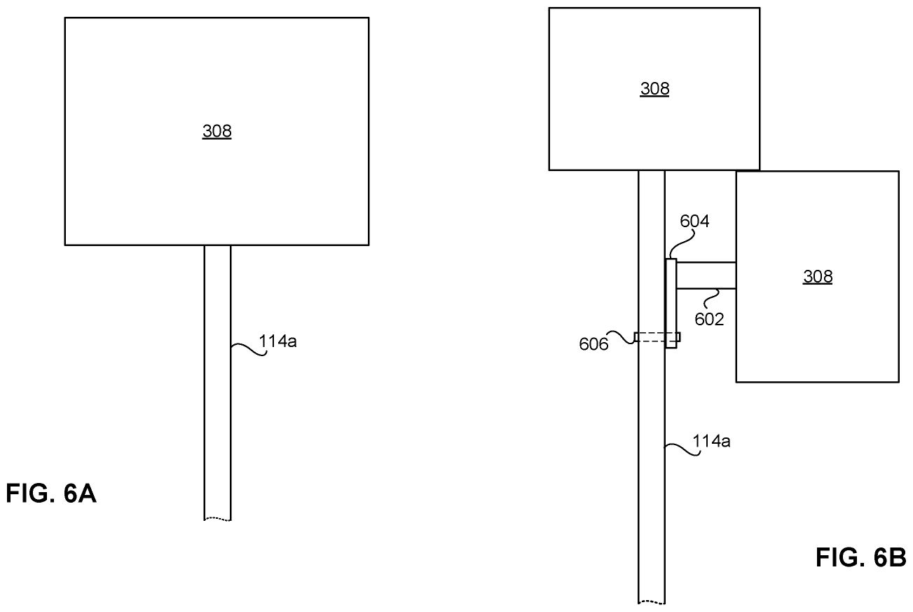

FIG. 6A illustrates the extender arm 114a extending directly from the motor 308, according to an embodiment. The motor 308 may directly drive the extendable agitator 114. This may ensure the maximum amount of power is transferred from the motor 308 to the extendable agitator 114. In some examples, the extender arm 114a may be the rotor of the motor 308.

FIG. 6B illustrates the extender arm 114a coupled to a rotor 602 of the motor 308 by a linear translation mechanism 604, according to an embodiment. In some examples of the handheld sanitizing device 100, the extendable agitator 114 may actuate along two degrees of freedom. Actuating the extendable agitator 114 along two degrees of freedom may increase mixing and/or turbulence in a fluid being stirred by the extendable agitator 114. Increased mixing and/or turbulence may lead to faster and/or more complete purification of the fluid being stirred by the extendable agitator 114.

The extender arm 114a may be coupled to the rotor 602 of the motor 308 by the linear translation mechanism 604. The linear translation mechanism 604 may translate rotation of the rotor 602 into linear motion of the extender arm 114a. The linear translation mechanism 604 may include a slot and a pin 606 attached to the extender arm 114a may be nested in the slot. As the linear translation mechanism 604 is rotated by the motor 308, the pin 606 may slide in the slot, causing the extender arm 114a to translate linearly perpendicular to the axis of rotation of the rotor 602. The extender arm 114a may further be coupled to a second instance of the motor 308, which may rotate the extender arm 114a. As the first and second instances of the motor 308 rotate, the extender arm 114a may rotate and translate linearly. The motors 308 may operate separately and/or in tandem. For example, the first instance of the motor 308 may be on while the second instance is off; the second instance of the motor 308 may be on while the first instance is off; both instances of the motor 308 may be on at the same time; and/or both instances of the motor 308 may be off at the same time.

A feature illustrated in one of the figures may be the same as or similar to a feature illustrated in another of the figures. Similarly, a feature described in connection with one of the figures may be the same as or similar to a feature described in connection with another of the figures. The same or similar features may be noted by the same or similar reference characters unless expressly described otherwise. Additionally, the description of a particular figure may refer to a feature not shown in the particular figure. The feature may be illustrated in and/or further described in connection with another figure.

Elements of processes (i.e. methods) described herein may be executed in one or more ways such as by a human, by a processing device, by mechanisms operating automatically or under human control, and so forth. Additionally, although various elements of a process may be depicted in the figures in a particular order, the elements of the process may be performed in one or more different orders without departing from the substance and spirit of the disclosure herein.

The foregoing description sets forth numerous specific details such as examples of specific systems, components, methods, and so forth, in order to provide a good understanding of several implementations. It will be apparent to one skilled in the art, however, that at least some implementations may be practiced without these specific details. In other instances, well-known components or methods are not described in detail or are presented in simple block diagram format in order to avoid unnecessarily obscuring the present implementations. Thus, the specific details set forth above are merely exemplary. Particular implementations may vary from these exemplary details and still be contemplated to be within the scope of the present implementations.

Related elements in the examples and/or embodiments described herein may be identical, similar, or dissimilar in different examples. For the sake of brevity and clarity, related elements may not be redundantly explained. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

It is to be understood that the foregoing description is intended to be illustrative and not restrictive. Many other implementations will be apparent to those of skill in the art upon reading and understanding the above description. The scope of the present implementations should, therefore, be determined with reference to the appended claims, along with the full scope of equivalents to which such claims are entitled.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions, and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every example. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, Abstract, and any other document and/or resource incorporated herein by reference.

As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted examples.

The Applicant(s) reserves the right to submit claims directed to combinations and sub-combinations of the disclosed examples that are believed to be novel and non-obvious. Examples embodied in other combinations and sub-combinations of features, functions, elements, and/or properties may be claimed through amendment of those claims or presentation of new claims in the present application or in a related application. Such amended or new claims, whether they are directed to the same example or a different example and whether they are different, broader, narrower, or equal in scope to the original claims, are to be considered within the subject matter of the examples described herein.Claims

1. A handheld sanitizing device, comprising:
an elongated housing, comprising:
a first end;
a second end opposite the first end;
a first cavity adjacent to the first end;
a second cavity adjacent to the second end;
a sanitizing light disposed at the first end and facing away from the elongated housing, wherein the sanitizing light is configured to kill biological contaminants in a substance or on a surface;
a removable cap that attaches to the first end and covers the sanitizing light;
a motor in the first cavity;
an extendable agitator that extends from the first end of the elongated housing, wherein the extendable agitator is configured to extend from the elongated housing and agitate a substance, the extendable agitator comprising:
an extender arm coupled to the motor, wherein the extender arm is:
extendable from the first cavity; and
retractable into the first cavity;
an agitator end coupled to the extender arm, wherein:
as the extender arm is retracted into the first cavity, the agitator end is positioned adjacent to the first end of the elongated housing; and
the agitator end is configured to stir a liquid substance as the motor actuates the extender arm;
a controller electronically coupled to the sanitizing light and the motor, wherein the controller controls operation of the sanitizing light or the motor;
a power port disposed at the second end of the elongated housing opposite the sanitizing light, wherein:
the power port is configured to receive an end of a power cable; and
the power port is electronically coupled to the sanitizing light, the motor, or the controller; and
the power port is configured to directly power the sanitizing light, the motor, or the controller.

2. The handheld sanitizing device of claim 1, further comprising a container that holds a liquid, wherein:
the elongated housing is configured to pass at least partially through a mouth of the container into a reservoir of the container;
the extendable agitator is configured to extend into the liquid and stir the liquid as the motor rotates the agitator end; and
the sanitizing light is configured to kill biological contaminants in the liquid as the extendable agitator stirs the liquid.

3. The handheld sanitizing device of claim 1, further comprising a solar charging panel, wherein:
the solar panel is physically coupled to the elongated housing;
a photosensitive surface of the solar panel faces outwards from the elongated housing;
the solar panel is electronically coupled to the sanitizing light, the motor, or the controller; and
the solar panel is configured to power the sanitizing light, the motor, or the controller.

4. The handheld sanitizing device of claim 1, the motor further comprising a rotor, wherein:
the extender arm is rotatably coupled to the rotor;
the extender arm is the rotor; or
the extender arm is coupled to the rotor by a linear translation mechanism that translates rotation of the rotor into linear motion of the extender arm.

5. The handheld sanitizing device of claim 1, further comprising an adapter that affixes to the elongated housing, wherein:
the adapter has a greater width than the elongated housing; and
the adapter is configured to rest against a mouth of a container and prevent the elongated housing from falling into the container.

6. The handheld sanitizing device of claim 1, wherein:
the sanitizing light is disposed in approximately a center of the first end; and
the extender arm is positioned adjacent to the sanitizing light and off-center from the center of the first end.

7. The handheld sanitizing device of claim 1, wherein:
the sanitizing light comprises a ring light adjacent to an edge of the first end of the elongated housing; and
the extender arm is positioned:
at a center of the first end; or
off-center from the center of the first end.

8. The handheld sanitizing device of claim 1, wherein:
the sanitizing light comprises a ring light; and
the agitator end nests within the sanitizing light as the agitator end is adjacent to the first end of the elongated housing.

9. The handheld sanitizing device of claim 1, wherein:
the agitator end comprises a ring; and
the agitator end encircles the sanitizing light as the agitator end is adjacent to the first end of the elongated housing.

10. A device, comprising:
a housing, wherein the housing has a greater length than a width of the housing;
a sanitizing light disposed at a first end of the housing, wherein:
the sanitizing light faces away from the housing; and
the sanitizing light is configured to at least partially sanitize a substance or a surface;
a removable cap that attaches to the first end of the housing and covers the sanitizing light;
a motor enclosed within the housing;
an extendable agitator that extends from the first end of the housing, the extendable agitator comprising:
an extender arm coupled to the motor, wherein:
the extender arm is actuatable by the motor; and
the extender arm is extendable from and retractable into the housing;
an agitator end coupled to the extender arm, wherein, as the extender arm is retracted into the housing, the agitator end is positioned adjacent to the first end of the housing; and
a battery enclosed within the housing, wherein the battery powers the sanitizing light or the motor.

11. The device of claim 10, wherein:
the removable cap comprises:
a first layer that comprises:
a first opening that aligns with the sanitizing light as the removable cap is attached to the housing; and
a ridge that attaches the removable cap to the housing; and
a second layer that comprises a second opening that corresponds to the first opening;
the first layer and the second layer are rotatably coupled to each other;
in a first position of the second layer relative to the first layer, the second opening is aligned with the first opening so that the sanitizing light is exposed; and
in a second position of the second layer relative to the first layer, the second layer covers the first opening so that the sanitizing light is covered.

12. The device of claim 10, further comprising a charging port electronically coupled to the battery, wherein:
the battery is rechargeable; and
the battery is rechargeable via the charging port.

13. The device of claim 10, further comprising:
a first switch and a second switch, wherein:
the first switch controls power to the sanitizing light; and
the second switch controls power to the motor; or
a single switch, wherein the single switch controls power to the sanitizing light and the motor.

14. The device of claim 10, wherein:
as the agitator is extended from the first end, the motor is engaged with the extender arm; or
as the agitator is retracted adjacent to the first end, the motor is disengaged from the extender arm.

15. The device of claim 10, further comprising a first switch and a second switch, wherein:
the first switch as positioned on an exterior of the housing;
the first switch controls power to the sanitizing light and the second switch;
the second switch controls power to the motor;
the second switch is automatically on as the agitator is extended a threshold length from the first end; and
the second switch is automatically off as the agitator is retracted to the housing less than the threshold length.

16. The device of claim 10, wherein the motor or the extender arm is configured to actuate the agitator end with two degrees of freedom as the agitator is extended from the first end.

17. A device, comprising:
a housing, wherein the housing has a greater length than a width of the housing;
a sanitizing light that emits light rays away from the housing, wherein the sanitizing light is configured to at least partially sanitize a substance or a surface;
a removable cap that attaches to the housing and covers the sanitizing light;
a motor coupled to or enclosed within the housing; and
an extendable agitator that extends from and retracts towards the housing, wherein:
the extendable agitator is operatively coupled to the motor; and
the extendable agitator is configured to stir a substance.

18. The device of claim 17, the extendable agitator further comprising an extender arm, wherein, as the extendable agitator is retracted towards the housing, the extender arm is positioned along a side of the housing.

19. The device of claim 17, the extendable agitator further comprising an extender arm and an agitator end, wherein the extender arm is coupled to the agitator end off-center from a center of a surface of the agitator end.

20. The device of claim 17, further comprising a power port electronically coupled to the sanitizing light and the motor, wherein the power port delivers power to the sanitizing light or the motor from an external power source.
